(19) United States
(12) Patent Application Publication
(10) Pub. No.: US 2011/0123579 A1
(43) Pub. Date: May 26, 2011

(54) Cosmetic Composition For Diminishing The Appearance Of Lines And Pores

(76) Inventors: Fatemeh Mohammadi, Hauppauge, NY (US); Lavinia C. Popescu, Jackson Heights, NY (US)

(21) Appl. No.: 12/771,650

(22) Filed: Apr. 30, 2010

Related U.S. Application Data
(63) Continuation-in-part of application No. 11/128,027, filed on May 12, 2005, now abandoned.

(51) Int. Cl. A61K 8/89; A61Q 19/00

(52) U.S. Cl. 424/401; 514/63

(57) ABSTRACT

Silicone-in-water emulsions and methods for reducing the appearance of lines, wrinkles and the size of pores in the skin are provided. The emulsions comprise at least one silicone, comprising at least one silicone elastomer, a silica, a film forming cosmetic patch component, and water.

### CROSS-REFERENCE TO RELATED APPLICATIONS

This application is a continuation in part of application Ser. No. 11/128,027, filed May 12, 2005, the contents of which are incorporated herein by reference.

### FIELD OF THE INVENTION

The invention relates to a cosmetic composition for topical application to the skin. More specifically, the invention relates to silicone-in-water emulsions which, on application to the skin, provide an immediate reduction in the appearance of lines and wrinkles and the size of pores in the skin.

### BACKGROUND OF THE INVENTION

As skin ages, particularly the skin on the face, it begins to exhibit fine lines and then wrinkles. Additionally, whether skin is young or old, it can exhibit enlarged pores, whether from a blemish condition or simply because as the skin ages, it becomes less resilient, and the pores, which have become enlarged through stretching of the skin, remain so. It is desirable that the skin appear to retain its youthful, lineless and poreless appearance.

Typical formulations for reducing the appearance of lines and wrinkles in older skins tend to be comprised of different components from those components typically found in formulations used for reducing the appearance of pore size. The latter compositions, which generally are intended for younger, oilier skins, are usually combined with drying and/or oil absorbing components, and, therefore, are not suitable for older skins exhibiting lines and wrinkles.

The present invention provides a cosmetic composition, which when applied to the skin, provides instant reduction in the appearance of fine lines, wrinkles, particularly in the eye and lip areas, and the size of pores, to create the appearance of a smoother, more flawless and more youthful looking complexion. The compositions of the invention can be applied alone, under makeup or over makeup, to provide the appearance of smoother, more youthful, beautiful skin.

### SUMMARY OF THE INVENTION

The present invention concerns silicone-in-water emulsions for reducing the appearance of one or more of fine lines, wrinkles and the size of pores in the skin, comprising (a) at least one silicone comprising at least one silicone elastomer; (b) at least one silica; (c) at least one film-forming component; and (d) water.

The invention also includes methods for reducing the appearance of one or more of fine lines, wrinkles and the size of pores in the skin, comprising applying to the skin in need of a reduction in the appearance of lines, wrinkles and/or pores, a cosmetically or dermatologically effective amount of a silicone-in-water emulsion comprising (a) at least one silicone comprising at least one silicone elastomer; (b) at least one silica: (c) at least one film-forming component; and (d) water.

### BRIEF DESCRIPTION OF THE DRAWINGS

FIG. 1 is a chart illustrating the percent reduction in the appearance of pores and lines and wrinkles immediately after the application to skin of a composition of the present invention.

FIG. 2 is a representation of a grey scale.

### DETAILED DESCRIPTION OF THE INVENTION

The novel cosmetic compositions of the invention are silicone-in-water formulations comprising (a) at least one silicone comprising at least one silicone elastomer, (b) at least one silica, (c) at least one film-forming component; and (d) water.

In one preferred embodiment of the invention the composition comprises a silicone-in-water emulsion consisting essentially of (a), (b), (c) and (d). By use of the term “consisting essentially of”, it is intended that the composition does not include any further component which would materially affect the desired properties imparted to the composition by the enumerated ingredients. The composition, when applied to the skin, particularly the facial skin, provides an immediate reduction in the appearance of fine lines, wrinkles and/or the size of the pores.

The silicone-in-water emulsion compositions of the invention are predominantly water, but may also include other solvents miscible with water, such as, for example, hydroalcohol, glycerin, and combinations thereof. Examples of acceptable hydroalcohols include, but are not limited to, ethanol, propanol or glycols such as butylene glycol, pentylene glycol and hexylene glycol. It is preferred that the polyol be a C₂-C₅ alcohol. Water may be present in the compositions of the invention in amount in the range of from about 5 to about 90 percent, more preferably from about 30 to about 70 percent, such as from about 40 to about 60 percent, based on the total weight of the composition.

Silicones (polyorganosiloxanes) useful in forming the silicone-in-water emulsions of the invention are polymers containing siloxane units independently selected from (R₃SiO_{0.5}), (R₂SiO), (RSiO_{1.5}), or (SiO₂) siloxy units, where R may be any monovalent organic group. When R is a methyl group in the (R₃SiO_{0.5}), (R₂SiO), (RSiO_{1.5}), or (SiO₂) siloxy units of an organopolysiloxane, the siloxy units are commonly referred to as M, D, T, and Q units respectively. These siloxy units can be combined in various manners to form cyclic, linear, or branched structures. Polyorganosiloxanes suitable for use in the compositions of the present invention are not particularly limited, and may be any cosmetically and dermatologically acceptable silicone- or siloxane-containing copolymer.

Cyclic silicones are one type of volatile silicone that may be used in the composition. Such silicones have the general formula:

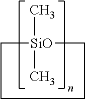

where n=3-6, preferably 4, 5, or 6.

Also suitable are linear volatile silicones, for example, those having the general formula:

(CH₃)₃Si—O—[Si(CH₃)₂—O]ₙ—Si(CH₃)₃

where n=0, 1, 2, 3, 4, or 5, preferably 0, 1, 2, 3, or 4.

Cyclic and linear volatile silicones are available from various commercial sources including Dow Corning Corporation and General Electric. The Dow Corning linear volatile silicones are sold under the tradenames Dow Corning 244, 245, 344, and 200 fluids. These fluids include hexamethyldisiloxane (viscosity 0.65 centistokes (abbreviated cst)), octamethyltrisiloxane (1.0 cst), decamethyltetrasiloxane (1.5 cst), dodecamethylpentasiloxane (2 cst) and mixtures thereof, with all viscosity measurements being at 25° C.

Suitable branched volatile silicones include alkyl trimethicones such as methyl trimethicone, a branched volatile silicone having the general formula:

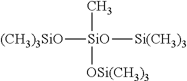

Methyl trimethicone may be purchased from Shin-Etsu Silicones under the tradename TMF-1.5, having a viscosity of 1.5 centistokes at 25° C.

Nonvolatile silicone oils, both water soluble and water insoluble, are also suitable for use in the composition. Such silicones preferably have a viscosity ranging from about greater than 5 to 800,000 cst, preferably 20 to 200,000 cst at 25° C. Suitable water insoluble silicones include amine functional silicones such as amodimethicone.

For example, such nonvolatile silicones may have the following general formula:

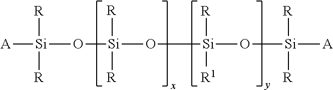

wherein R and R′ are each independently C₁₋₃₀ straight or branched chain, saturated or unsaturated alkyl, phenyl or aryl, trialkylsiloxy, and x and y are each independently 1-1,000,000; with the proviso that there is at least one of either x or y, and A is alkyl siloxy endcap unit. Preferred is where A is a methyl siloxy endcap unit; in particular trimethylsiloxy, and R and R′ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, diphenyl dimethicone, phenyl trimethicone, or trimethylsiloxyphenyl dimethicone. Other examples include alkyl dimethicones such as cetyl dimethicone, and the like wherein at least one R is a fatty alkyl (C₁₂, C₁₄, C₁₆, C₁₈, C₂₀, or C₂₂), and the other R is methyl, and A is a trimethylsiloxy endcap unit, provided such alkyl dimethicone is a pourable liquid at room temperature. Phenyl trimethicone can be purchased from Dow Corning Corporation under the tradename 556 Fluid. Trimethylsiloxyphenyl dimethicone can be purchased from Wacker-Chemie under the tradename PDM-1000. Cetyl dimethicone, also referred to as a liquid silicone wax, may be purchased from Dow Corning as Fluid 2502, or from DeGussa Care & Surface Specialties under the trade names Abil Wax 9801, or 9814.

Silicone elastomers, silicone gums, silicone waxes, and linear silicones may also be useful in the present invention.

Silicone elastomers suitable for use in the compositions of the invention include those that are formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene; or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally cure in the presence of an organoperoxide catalyst.

One type of elastomer that may be suitable is prepared by addition reaction-curing an organopolysiloxane having at least 2 lower alkenyl groups in each molecule or an alpha-omega diene; and an organopolysiloxane having at least 2 silicon-bonded hydrogen atoms in each molecule; and a platinum-type catalyst. While the lower alkenyl groups such as vinyl, can be present at any position in the molecule, terminal olefinic unsaturation on one or both molecular terminals is preferred. The molecular structure of this component may be straight chain, branched straight chain, cyclic, or network. These organopolysiloxanes are exemplified by methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl)polysiloxanes, and dimethylvinylsiloxy-terminated dimethylsiloxane-methyl(3,3-trifluoropropyl)siloxane copolymers, decadiene, octadiene, heptadiene, hexadiene, pentadiene, or tetradiene, or tridiene.

Curing proceeds by the addition reaction of the silicon-bonded hydrogen atoms in the dimethyl methylhydrogen siloxane, with the siloxane or alpha-omega diene under catalysis using the catalyst mentioned herein. To form a highly crosslinked structure, the methyl hydrogen siloxane must contain at least 2 silicon-bonded hydrogen atoms in each molecule in order to optimize function as a crosslinker.

The catalyst used in the addition reaction of silicon-bonded hydrogen atoms and alkenyl groups, and is concretely exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

Examples of suitable silicone elastomers for use in the compositions of the invention may be in the powder form, or dispersed or solubilized in solvents such as volatile or non-volatile silicones, or silicone compatible vehicles such as paraffinic hydrocarbons or esters. Examples of silicone elastomer powders include vinyl dimethicone/methicone silsesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Corning's DC 9506.

Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the tradenames KSG-15, 16, 18. KSG-15 has the CTFA name cyclopentasiloxane/dimethicone/vinyl dimethicone crosspolymer. KSG-18 has the INCI name phenyl trimethicone/dimethicone/phenyl vinyl dimethicone crossopolymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo KK, each of which are herein incorporated by reference in its entirety.

Preferred silicone elastomers for use in the present invention, include, but are not limited to, dimethicone//dimethicone/vinyl dimethicone crosspolymer, phenyl trimethicone//dimethicone/phenyl vinyl dimethicone crosspolymer, cyclopentasiloxane//dimethicone/vinyl dimethicone crosspolymer, dimethicone/polysilicone-11, cyclopentasiloxane/polysilicone-11, phenyl trimethicone/polysilicone-11, cyclomethicone/polysilicone-11/petrolatum, cyclomethicone/polysilicone-11, stearoxy methicone/dimethicone copolymer, a mixture of water/propylene glycol/cyclomethicone D5/polysilicone-11/butylenes glycol/glycerine/isohexadecane/ammonium polyacryloyldimethyl, a mixture of butylenes glycol/water/dimethicone/polysilicone-11/glycerin/isohexadecane/ammonium polyacryloyldimethyl taurate/retinyl palmitate, dimethicone/polysilicone-11/cyclomethicone/trimethylsiloxysilicate, and silicone resin-coated elastomeric core material.

In one preferred embodiment of the present invention the silicone elastomer comprises silicon resin-coated elastomeric core materials consisting of a spherical silicone rubber core chemically bonded to an outer shell of silicone resin. These materials swell in silicone fluids such as cyclomethicone and dimethicone. The silicon resin shell confers a soft, cushioning feel, and the elastomeric core provides slip lubricity. Together, these materials provide the compositions of the invention with properties which impart a unique silky texture to the skin to which the composition is applied. Most preferred for use are hybrid silicone powders for example, vinyl dimethicone/methicone silsesquioxane crosspolymers, such as KSP-100, 101, 102, and 105, available from Shin Etsu Chemical Company., Ltd. (Tokyo, Japan). Particularly preferred is KSP-100.

Also suitable for use in the emulsions of the present invention are one or more silicone gums. The term “gum” means a silicone polymer having a degree of polymerization sufficient to provide a silicone having a gum-like texture. In certain cases the silicone polymer forming the gum may be crosslinked. The silicone gum typically has a viscosity ranging from about 500,000 to 100 million cst at 25° C., preferably from about 600,000 to 20 million, more preferably from about 600,000 to 12 million cst. All ranges mentioned herein include all subranges, e.g. 550,000; 925,000; 3.5 million. The silicone gums that are used in the compositions include, but are not limited to, those of the general formula wherein:

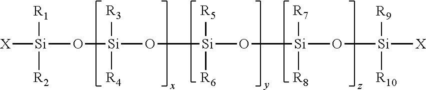

R₁ to R₉ are each independently an alkyl having 1 to 30 carbon atoms, aryl, or aralkyl; and X is OH or a C₁₋₃₀ alkyl, or vinyl; and wherein x, y, or z may be zero with the proviso that no more than two of x, y, or z are zero at any one time, and further that x, y, and z are such that the silicone gum has a viscosity of at least about 500,000 cst, ranging up to about 100 million centistokes at 25° C. Preferred is where R is methyl or OH.

Such silicone gums may be purchased in pure form from a variety of silicone manufacturers including Wacker-Chemie or Dow Corning, and the like. Such silicone gums include those sold by Wacker-Belsil under the trade names CM3092, Wacker-Belsil 1000, or Wacker-Belsil DM 3096. A silicone gum where X is OH, also referred to as dimethiconol, is available from Dow Corning Corporation under the trade name 1401. The silicone gum may also be purchased in the form of a solution or dispersion in a silicone compatible vehicle such as volatile or nonvolatile silicone. An example of such a mixture may be purchased from Barnet Silicones under the HL-88 tradename, having the INCI name dimethicone.

Silicone waxes useful in the emulsions of the present invention are typically referred to as alkyl silicone waxes which are semi-solids or solids at room temperature. The term “alkyl silicone wax” means a polydimethylsiloxane having a substituted long chain alkyl (such as C₁₆ to ₃₀) that confers a semi-solid or solid property to the siloxane. Examples of such silicone waxes include stearyl dimethicone, which may be purchased from DeGussa Care & Surface Specialties under the tradename Abil Wax 9800 or from Dow Corning under the tradename 2503. Another example is bis-stearyl dimethicone, which may be purchased from Gransil Industries under the tradename Gransil A-18, Gransil EP-LS, Gransil DM-5, behenyl dimethicone, behenoxy dimethicone.

Also suitable are various types of polymeric compounds such as silicone polyamides. The term silicone polyamide means a polymer comprised of silicone monomers and monomers containing amide groups as further described herein. The silicone polyamide preferably comprises moieties of the general formula:

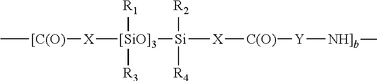

X is a linear or branched alkylene having from about 1-30 carbon atoms; R₁, R₂, R₃, and R₄ are each independently C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

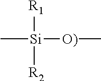

### and Y is:

(a) a linear or branched alkylene having from about 1-40 carbon atoms which may be substituted with:
   (i) one or more amide groups having the general formula R₁CONR₁, or
   (ii) C₅₋₆ cyclic ring, or
   (iii) phenylene which may be substituted with one or more C₁₋₁₀ alkyl groups, or
   (iv) hydroxy, or
   (v) C₃₋₈ cycloalkane, or
   (vi) C₁₋₂₀ alkyl which may be substituted with one or more hydroxy groups, or
   (vii) C₁₋₁₀ alkyl amines; or
(b) TR₅R₆R₇
   wherein R₅, R₆, and R₇, are each independently a C₁₋₁₀ linear or branched alkylenes, and T is CR₈ wherein R₈ is hydrogen, a trivalent atom N, P, or Al, or a C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

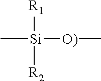

Preferred is where R₁, R₂, R₃, and R₄ are C₁₋₁₀, preferably methyl; and X and Y is a linear or branched alkylene. Preferred are silicone polyamides having the general formula

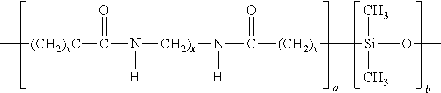

wherein a and b are each independently sufficient to provide a silicone polyamide polymer having a melting point ranging from about 60 to 120° C., and a molecular weight ranging from about 40,000 to 500,000 Daltons. One type of silicone polyamide that may be used in the compositions of the invention may be purchased from Dow Corning Corporation under the tradename Dow Corning 2-8178 gellant which has the CTFA name nylon-611/dimethicone copolymer which is sold in a composition containing PPG-3 myristyl ether.

Also suitable are polyamides such as those purchased from Arizona Chemical under the tradenames Uniclear and Sylvaclear. Such polyamides may be ester terminated or amide terminated. Examples of ester terminated polyamides include, but are not limited to those having the general formula:

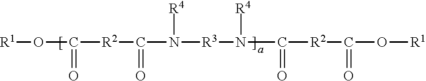

wherein n denotes a number of amide units such that the number of ester groups ranges from about 10% to 50% of the total number of ester and amide groups; each R₁ is independently an alkyl or alkenyl group containing at least 4 carbon atoms; each R₂ is independently a C₄₋₄₂ hydrocarbon group, with the proviso that at least 50% of the R₂ groups are a C₃₀₋₄₂ hydrocarbon; each R₃ is independently an organic group containing at least 2 carbon atoms, hydrogen atoms and optionally one or more oxygen or nitrogen atoms; and each R₄ is independently a hydrogen atom, a C₁₋₁₀ alkyl group or a direct bond to R₃ or to another R₄, such that the nitrogen atom to which R₃ and R₄ are both attached forms part of a heterocyclic structure defined by R₄—N—R₃, with at least 50% of the groups R₄ representing a hydrogen atom.

General examples of ester and amide terminated polyamides that may be used include those sold by Arizona Chemical under the tradenames Sylvaclear A200V or A2614V, both having the CTFA name ethylenediamine/hydrogenated dimer dilinoleate copolymer/bis-di-C₁₄₋₁₈ alkyl amide; Sylvaclear AF1900V; Sylvaclear C75V having the CTFA name bis-stearyl ethylenediamine/neopentyl glycol/stearyl hydrogenated dimer dilinoleate copolymer; Sylvaclear PA1200V having the CTFA name Polyamide-3; Sylvaclear PE400V; Sylvaclear WF1500V; or Uniclear, such as Uniclear 100VG having the INCI name ethylenediamine/stearyl dimer dilinoleate copolymer; or ethylenediamine/stearyl dimer ditallate copolymer. Other examples of suitable polyamides include those sold by Henkel under the Versamid trademark (such as Versamid 930, 744, 1655), or by Olin Mathieson Chemical Corp. under the brand name Onamid S or Onamid C.

The silicones may be present in the composition of the present invention in amounts in the range of about 5 to about 40 percent, preferably in the range of about 10 to about 30 weight percent, and more preferably, in the range of about 10 to about 20 weight percent, based on the total weight of the composition. The silicone elastomers may comprise up to 100 percent of the silicone component.

The compositions of the present invention also include silica, including coated or encapsulated silicas.

The silica may be any type of silica suitable for cosmetic use, such as silica, silica silylate (hydrophobic silica), and silica provided in bead form. Examples of silicas suitable for use in the present invention include, but are not limited to, silica microspheres, such as MSS-500/3; Spheron P-1500; Cab-o-sil M7-D; silica beads SB-700; Monoveil and Monoveil Spectra T. Monoveil and Monoveil Spectra T, which are available from Ried International Corporation (Brentwood, N.Y.), are particularly preferred for use in the compositions of the present invention. Due to their multi-layered and spherical forms, visible light rays are refracted inside the sphere's layers and reflected at various angles. The light scattering effect provided blurs fine lines, wrinkles, and pores while also providing transparency.

Coated or encapsulated silicas are also useful in the compositions of the present invention, and include, for example, silica/triethoxycaprylsilane, silica/methicone, silica/dimethicone, silica/isononyl isonanoate, silica/octyldodecyl stearoyl stearate/isononyl isononanoate, silica/methoxy amodimethicone silsesquioxane copolymer, silica/ultramarines/kaolin/PVP/water, silica silylate, and silica/hexyldecyldiisocyanate/trimethylol hexylactone crosspolymer.

The silica component may be present in the compositions of the present invention in amounts in the range of from about 1 to about 40 weight percent, preferably in the range of from about 5 to about 30 weight percent, and more preferably in the range of from about 5 to about 10 weight percent, based on the total weight of the composition.

In one preferred embodiment of the present invention, the emulsion comprises a combination of a spherical, preferably a multilayered, silica and a coated or encapsulated silica, such as silica/hexyldecyldiisocyanate/trimethylol hexylactone crosspolymer.

Compositions of the present invention further include a film-forming component. The film-forming component may be any cosmetically or dermatologically acceptable film-forming material which will act as a cosmetic patch to enhance the adherence of the powder components to the skin while minimizing water loss from cutaneous tissue. In preferred embodiments of the present invention, the cosmetic patch includes algin, serine and atelocollagen. Such a patch component is available, as the Moisturizing Marine MICROPATCH®, from Coletica (Northport, N.Y.). It is believed that the collagen and the alginate in the patch are capable of associating with the skin, and together forming a filmogenic micronetwork which acts to prevent the evaporation of moisture from the skin, and which holds serine, an amino acid and synthesis precursor of ceramides, which are naturally occurring skin lipids. The serine is slowly released from the network into the skin for extended moisturization. It is believed that the release of serine into the upper layers of the epidermis stimulates the production of ceramides in the skin. A further example of a film-forming component suitable for use in the emulsions of the present invention is a water-soluble polymer of 2-methacryloyloxy ethyl phosphorylcholine (MPC), available as Lipidure® from NOF Corporation, Japan.

The film-forming component may be present in the compositions of the invention in the range of about 0.01 to about 20 weight percent, preferably in the range of about 0.1 to about 10 weight percent, and more preferably, in the range of about 0.5 to about 5 weight percent, such as in the range of from about 1 to about 3 percent, based on the total weight of the composition.

The compositions of the invention are substantially oil-free formulations. By “substantially oil-free”, it is intended that, if oil is present in the composition, the amount of oil is no more than 5 weight percent, based on the total weight of the composition. Oils include silicones, esters, vegetable oils, synthetic oils, for example, paraffinic hydrocarbons, esters, hydrocarbon oils, glyceryl esters of fatty acids, and so forth. Oils may be volatile or nonvolatile, and are generally liquid at room temperature. The term “volatile” means that the oil has a measurable vapor pressure or a vapor pressure of at least about 2 mm. of mercury at 20° C. The term “nonvolatile” means that the oil has a vapor pressure of less than about 2 mm. of mercury at 20° C. Whereas the presence of oils in the compositions of the invention would solubilize the other components and destroy the optical effect of the emulsions, the silicones, on the other hand, do not solubilize the other ingredients and therefore allow the powder components to deposit in lines and pores in the skin.

While not wishing to be bound by any particular theory, the Applicants believe that when the compositions of the present invention are applied to the skin, the silica, and silicone elastomer particles, such as particles of silicone resin coated silicone elastomeric core material, deposit in the pores and fine lines, and the film forming component forms a patch over the powder components, sealing them against the skin. The rubbery silicone material swells in silicone fluids, filling lines and pores, while the silica, having an index of refraction near that of the skin, imparts a mattifying effect in the more reflective lines and pores, with the result that the lines and pores become less reflective, similar to the relatively more matte surrounding skin, and therefore, less visible.

In addition to water, the aqueous phase of the compositions of the present invention may contain one or more aqueous phase structuring agents, that is, an agent that increases the viscosity or, or thickens, the aqueous phase of the composition. This is particularly desirable when the composition is in the form of a serum or gel. Suitable ranges of aqueous phase structuring agent, if present, are from about 0.01 to 30%, preferably from about 0.1 to 20%, more preferably from about 0.5 to 15% by weight of the total composition. Examples of such agents include various acrylate based thickening agents, natural or synthetic gums, polysaccharides, and the like.

Polysaccharides may be suitable aqueous phase thickening agents. Examples of such polysaccharides include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, and so on.

Also suitable are different types of synthetic polymeric thickeners. One type includes acrylic polymeric thickeners comprised of monomers A and B wherein A is selected from the group consisting of acrylic acid, methacrylic acid, and mixtures thereof; and B is selected from the group consisting of a C₁₋₂₂ alkyl acrylate, a C₁₋₂₂ alky methacrylate, and mixtures thereof are suitable. In one embodiment the A monomer comprises one or more of acrylic acid or methacrylic acid, and the B monomer is selected from the group consisting of a C₁₋₁₀, most preferably C₁₋₄ alkyl acrylate, a C₁₋₁₀, most preferably C₁₋₄ alkyl methacrylate, and mixtures thereof. Most preferably the B monomer is one or more of methyl or ethyl acrylate or methacrylate. The acrylic copolymer may be supplied in an aqueous solution having a solids content ranging from about 10-60%, preferably 20-50%, more preferably 25-45% by weight of the polymer, with the remainder water. The composition of the acrylic copolymer may contain from about 0.1-99 parts of the A monomer, and about 0.1-99 parts of the B monomer. Acrylic polymer solutions include those sold by Seppic, Inc., under the tradename Capigel.

Also suitable are acrylic polymeric thickeners that are copolymer of A, B, and C monomers wherein A and B are as defined above, and C has the general formula:

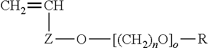

wherein Z is —(CH₂)ₘ; wherein m is 1-10, n is 2-3, o is 2-200, and R is a C₁₀₋₃₀ straight or branched chain alkyl. Examples of the secondary thickening agent above, are copolymers where A and B are defined as above, and C is CO, and wherein n, o, and R are as above defined. Examples of such secondary thickening agents include acrylates/steareth-20 methacrylate copolymer, which is sold by Rohm & Haas under the tradename Acrysol ICS-1.

Also suitable are acrylate based anionic amphiphilic polymers containing at least one hydrophilic unit and at least one allyl ether unit containing a fatty chain. Preferred are those where the hydrophilic unit contains an ethylenically unsaturated anionic monomer, more specifically a vinyl carboxylic acid such as acrylic acid, methacrylic acid or mixtures thereof, and where the allyl ether unit containing a fatty chain corresponds to the monomer of formula

CH₂═CR′CH₂OBₙR

in which R′ denotes H or CH₃, B denotes the ethylenoxy radical, n is zero or an integer ranging from 1 to 100, R denotes a hydrocarbon radical selected from alkyl, arylalkyl, aryl, alkylaryl and cycloalkyl radicals which contain from 8 to 30 carbon atoms, preferably from 10 to 24, and even more particularly from 12 to 18 carbon atoms. More preferred in this case is where R′ denotes H, n is equal to 10 and R denotes a stearyl (C18) radical. Anionic amphiphilic polymers of this type are described and prepared in U.S. Pat. Nos. 4,677,152 and 4,702,844, both of which are hereby incorporated by reference in their entirety. Among these anionic amphiphilic polymers, polymers formed of 20 to 60% by weight acrylic acid and/or methacrylic acid, of 5 to 60% by weight lower alkyl methacrylates, of 2 to 50% by weight allyl ether containing a fatty chain as mentioned above, and of 0 to 1% by weight of a crosslinking agent which is a well-known copolymerizable polyethylenic unsaturated monomer, for instance diallyl phthalate, allyl (meth)acrylate, divinylbenzene, (poly)ethylene glycol dimethacrylate and methylenebisacrylamide. One commercial example of such polymers are crosslinked terpolymers of methacrylic acid, of ethyl acrylate, of polyethylene glycol (having 10 EO units) ether of stearyl alcohol or steareth-10, in particular those sold by the company Allied Colloids under the names SALCARE SC80 and SALCARE SC90, which are aqueous emulsions containing 30% of a crosslinked terpolymer of methacrylic acid, of ethyl acrylate and of steareth-10 alkyl ether (40/50/10).

Also suitable are acrylate copolymers such as Polyacrylate-3 which is a copolymer of methacrylic acid, methylmethacrylate, methylstyrene isopropylisocyanate, and PEG-40 behenate monomers; Polyacrylate-10 which is a copolymer of sodium acryloyldimethyltaurate, sodium acrylate, acrylamide and vinyl pyrrolidone monomers; or Polyacrylate-11, which is a copolymer of sodium acryloyldimethylacryloyldimethyl taurate, sodium acrylate, hydroxyethyl acrylate, lauryl acrylate, butyl acrylate, and acrylamide monomers.

Also suitable are crosslinked acrylate based polymers where one or more of the acrylic groups may have substituted long chain alkyl (such as 6-40, 10-30, and the like) groups, for example acrylates/C₁₀₋₃₀ alkyl acrylate crosspolymer which is a copolymer of C₁₀₋₃₀ alkyl acrylate and one or more monomers of acrylic acid, methacrylic acid, or one of their simple esters crosslinked with the allyl ether of sucrose or the allyl ether of pentaerythritol. Such polymers are commonly sold under the Carbopol or Pemulen tradenames and have the CTFA name carbomer.

One particularly suitable type of aqueous phase thickening agent are acrylate based polymeric thickeners sold by Clariant under the Aristoflex trademark such as Aristoflex AVC, which is ammonium acryloyldimethyltaurate/VP copolymer; Aristoflex AVL which is the same polymer has found in AVC dispersed in mixture containing caprylic/capric triglyceride, trilaureth-4, and polyglyceryl-2 sesquiisostearate; or Aristoflex HMB which is ammonium acryloyldimethyltaurate/beheneth-25 methacrylate crosspolymer, and the like.

Also suitable as the aqueous phase thickening agents are various polyethylene glycols (PEG) derivatives where the degree of polymerization ranges from 1,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CFTA names polyglycerin-20, polyglycerin-40, and the like.

The composition may contain one or more surfactants. Such surfactants may be silicone or organic based. The surfactants will aid in the formation of stable emulsions. If present, the surfactant may range from about 0.001 to 30%, preferably from about 0.005 to 25%, more preferably from about 0.1 to 20% by weight of the total composition.

Suitable silicone surfactants include polyorganosiloxane polymers that have amphiphilic properties, for example contain hydrophilic radicals and lipophilic radicals. These silicone surfactants may be liquids or solids at room temperature.

One type of silicone surfactant that may be used is generally referred to as dimethicone copolyol or alkyl dimethicone copolyol. This surfactant is either a water-in-oil or oil-in-water surfactant having an Hydrophile/Lipophile Balance (HLB) ranging from about 2 to 18. Preferably the silicone surfactant is a nonionic surfactant having an HLB ranging from about 2 to 12, preferably about 2 to 10, most preferably about 4 to 6. The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof. The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof.

One type of suitable silicone surfactant has the general formula:

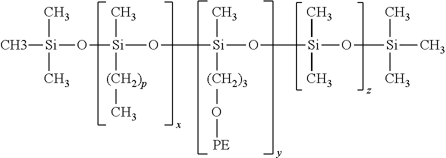

wherein p is 0-40 (the range including all numbers between and subranges such as 2, 3, 4, 13, 14, 15, 16, 17, 18, etc.), and PE is (—C₂H₄O)ₐ—(—C₃H₆O)_{b}—H wherein a is 0 to 25, b is 0-25 with the proviso that both a and b cannot be 0 simultaneously, x and y are each independently ranging from 0 to 1 million with the proviso that they both cannot be 0 simultaneously. In one preferred embodiment, x, y, z, a, and b are such that the molecular weight of the polymer ranges from about 5,000 to about 500,000, more preferably from about 10,000 to 100,000, and is most preferably approximately about 50,000 and the polymer is generically referred to as dimethicone copolyol.

One type of silicone surfactant is wherein p is such that the long chain alkyl is cetyl or lauryl, and the surfactant is called, generically, cetyl dimethicone copolyol or lauryl dimethicone copolyol respectively.

In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or an ether such as methyl ether, ethyl ether, propyl ether, butyl ether, and the like.

Examples of silicone surfactants are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; KF-6038 sold by Shin-Etsu Silicones having the CTFA name lauryl PEG-9 polydimethylsiloxyethyl dimethicone, or KF-6100 sold by Shin-Etsu Silicones having the CTFA name polyglyceryl-3 disiloxane dimethicone.

Also suitable are various types of crosslinked silicone surfactants that are often referred to as emulsifying elastomers. They are typically prepared as set forth above with respect to the section “silicone elastomers” except that the silicone elastomers will contain at least one hydrophilic moiety such as polyoxyalkylenated groups. Typically these polyoxyalkylenated silicone elastomers are crosslinked organopolysiloxanes that may be obtained by a crosslinking addition reaction of diorganopolysiloxane comprising at least one hydrogen bonded to silicon and of a polyoxyalkylene comprising at least two ethylenically unsaturated groups. In at least one embodiment, the polyoxyalkylenated crosslinked organo-polysiloxanes are obtained by a crosslinking addition reaction of a diorganopolysiloxane comprising at least two hydrogens each bonded to a silicon, and a polyoxyalkylene comprising at least two ethylenically unsaturated groups, optionally in the presence of a platinum catalyst, as described, for example, in U.S. Pat. No. 5,236,986 and U.S. Pat. No. 5,412,004, U.S. Pat. No. 5,837,793 and U.S. Pat. No. 5,811,487, the contents of which are incorporated by reference.

Polyoxyalkylenated silicone elastomers that may be used include those sold by Shin-Etsu Silicones under the names KSG-21, KSG-20, KSG-30, KSG-31, KSG-32, KSG-33; KSG-210 which is dimethicone/PEG-10/15 crosspolymer dispersed in dimethicone; KSG-310 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer.

Also suitable are polyglycerolated silicone elastomers like those disclosed in PCT/WO 2004/024798, which is hereby incorporated by reference in its entirety. Such elastomers include Shin-Etsu's KSG series, such as KSG-710 which is dimethicone/polyglycerin-3 crosspolymer dispersed in dimethicone; or lauryl dimethicone/polyglycerin-3 crosspolymer dispersed in a variety of solvent such as isododecane, dimethicone, triethylhexanoin, sold under the Shin-Etsu tradenames KSG-810, KSG-820, KSG-830, or KSG-840. Also suitable are silicones sold by Dow Corning under the tradenames 9010 and DC9011.

One preferred crosslinked silicone elastomer emulsifier is dimethicone/PEG-10/15 crosspolymer, which provides excellent aesthetics due to its elastomeric backbone, but also surfactancy properties.

The composition may comprise one or more nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Preferred non-ionic surfactants include laureth-4, laureth-12 and laureth-23.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Examples include polymeric alkylene glycols reacted with glyceryl fatty acid esters such as PEG glyceryl oleates, PEG glyceryl stearate; or PEG polyhydroxyalkanotes such as PEG dipolyhydroxystearate wherein the number of repeating ethylene glycol units ranges from 3 to 1000.

Also suitable as nonionic surfactants are formed by the reaction of a carboxylic acid with an alkylene oxide or with a polymeric ether. The resulting products have the general formula:

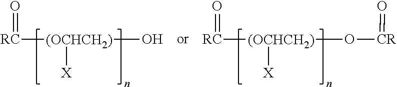

where RCO is the carboxylic ester radical, X is hydrogen or lower alkyl, and n is the number of polymerized alkoxy groups. In the case of the diesters, the two RCO-groups do not need to be identical. Preferably, R is a C6-30 straight or branched chain, saturated or unsaturated alkyl, and n is from 1-100.

Monomeric, homopolymeric, or block copolymeric ethers are also suitable as nonionic surfactants. Typically, such ethers are formed by the polymerization of monomeric alkylene oxides, generally ethylene or propylene oxide. Such polymeric ethers have the following general formula:

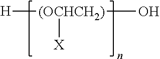

wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C6-30, preferably C12-22 fatty acids. Examples of such ingredients include Polysorbates 20-85, sorbitan oleate, sorbitan sesquioleate, sorbitan palmitate, sorbitan sesquiisostearate, sorbitan stearate, and so on.

Certain types of amphoteric, zwitterionic, or cationic surfactants may also be used in the compositions. Descriptions of such surfactants are set forth in U.S. Pat. No. 5,843,193, which is hereby incorporated by reference in its entirety.

It may also be desirable to include one or more humectants in the composition. If present, such humectants may range from about 0.001 to 25%, preferably from about 0.005 to 20%, more preferably from about 0.1 to 15% by weight of the total composition. Examples of suitable humectants include glycols, sugars, and the like. Suitable glycols are in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Also suitable is urea. Preferably, the humectants used in the composition of the invention are C₁₋₆, preferably C₂₋₄ alkylene glycols, most particularly butylene glycol.

It may be desirable to include one or more botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including yeast ferment extract, Padina Pavonica extract, thermus thermophilis ferment extract, camelina sativa seed oil, boswellia serrata extract, olive extract, Aribodopsis Thaliana extract, Acacia Dealbata extract, Acer Saccharinum (sugar maple), acidopholus, acorns, aesculus, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza Glabra, Salix Nigra, Macrocycstis Pyrifera, Pyrus Malus, Saxifraga Sarmentosa, Vitis Vinifera, Morus Nigra, Scutellaria Baicalensis, Anthemis Nobilis, Salvia Sclarea, Rosmarinus Officianalis, Citrus Medica Limonum, Panax Ginseng, Siegesbeckia Orientalis, Fructus Mume, Ascophyllum Nodosum, Bifida Ferment lysate, Glycine Soja extract, Beta Vulgaris, Haberlea Rhodopensis, Polygonum Cuspidatum, Citrus Aurantium Dulcis, Vitis Vinifera, Selaginella Tamariscina, Humulus Lupulus, Citrus Reticulata Peel, Punica Granatum, Asparagopsis, Curcuma Longa, Menyanthes Trifoliata, Helianthus Annuus, Hordeum Vulgare, Cucumis Sativus, Evernia Prunastri, Evernia Furfuracea, and mixtures thereof. Certain embodiments of the present invention may include anti-acne agents, such as, for example, Laminaria saccharina extract, 10-hydroxydecanoic acid, and phytosphingosine; whitening agents, such as for example, licorice root extract, ferulic acid and hinokito; firming ingredients, such as, for example, vitamin C and/or derivatives thereof, such as aminopropyl ascorbyl phosphate, glycerylpolymethacrylate/PEG-8/palmitoyloligopeptide and acetylhexapeptide-3 (argireline); and lifting agents, such as, for example, sweet almond seed extract, pea extract and algae extract/pullulan.

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, caprylyl glycol, biguanide derivatives, phenoxyethanol, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and the like. In one preferred embodiment the composition is free of parabens.

The compositions of the invention may contain vitamins and/or coenzymes, as well as antioxidants. If so, 0.001-10%, preferably 0.01-8%, more preferably 0.05-5% by weight of the total composition is suggested. Suitable vitamins include ascorbic acid and derivatives thereof such as ascorbyl palmitate, tetrahexydecyl ascorbate, and so on; the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are retinol palmitate, retinol. retinoic acid, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, phenoxyethanol, sodium dehyroacetate, and so forth.

The compositions of the subject invention also may include skin conditioning agents, such as hydrogenated lecithin, polypropylene terephthalate, and butylene glycol, and silicones, such as polyglycerin modified silicones, for example, polyglyceryl-3 disiloxane dimethicone, for example, KF-6100 (Shin-Etsu, Tokyo, Japan) which functions as a skin conditioner but also demonstrates a light scattering property and contributes to achieving a soft focus effect on the skin. Also suitable as conditioning agents are Butyrospermum parkii (shea butter) extract, Helianthus annuus (sunflower) seedcake, Sigesbeckia orientalis (St. Pauls's Wort) extract, Anthemis nobilis (chamomile), Cucumis melo (melon) fruit extract, Hordeum vulgare (barley) extract, Cucumis sativus (cucumber) fruit extract, Polygonum cuspidatum root extract, Chamomilla recutita (Matricaria), Camellia sinensis (white tea) leaf extract, and Rosmarinum officianalis (rosemary) leaf extract.

Compositions of the invention may be utilized in various topical forms, including creams, lotions, serums, gels and cream gels.

The following non-limiting examples further illustrate the embodiments of the invention.

### EXAMPLES

### Example 1

### Composition of the Invention—Lotion

TABLE 1

| | WEIGHT |
|---|---|
| MATERIALS | PERCENT |
| sequence 1 | |
| purified water | 52.25 |
| ammonium acrylodimethyltaurate/VP copolymer | 0.70 |
| sequence 2 | |
| disodium EDTA | 0.05 |
| sequence 3 | |
| butylene glycol | 0.50 |
| polyglyceryl-3 disiloxane dimethicone | 1.00 |
| vinyl dimethicone/methicone silsesquioxane crosspolymer | 10.00 |
| hydrogenated lecithin; | 1.50 |
| hexyldecyldiisocyanate/trimethylol hexylactone | 10.00 |
| crosspolymer//silica | |
| dimethicone crosspolymer-3/isododecane | 2.00 |
| denatured alcohol | 20.00 |
| purified water/butylene glycol/algin/atelocollagen/serine | 2.00 |
| TOTAL | 100.00 |

The sequence 1 ingredients are combined and gently mixed for about 20 minutes at room temperature until the mixture forms a clear gel. The temperature of the mixture is gradually increased until a temperature of 70° C. is reached at which time the sequence 2 component is introduced to the mixture. Sequence 3 ingredients are mixed together separately, and the temperature of the mixture is gradually increased to 75° C. The mixture of sequence 3 ingredients is introduced to the mixture with stirring. The combined mixture is gradually cooled to 35° C. and mixed until homogenized.

### Example 2

The composition of Example 1 is tested on sixteen women (panelists) having large pores and having lines in the eye area. The product is applied one time on the day of the test. A measured amount (approximately 0.5 cc) is dispensed from a syringe to the hands of the subject by the investigator. The subject then is instructed to apply the product to the skin on the face with the fingertips.

Optical Reduction in the Appearance of Lines and Wrinkles

Prior to, and immediately after product application, the appearance of lines and wrinkles on each panelist's skin is assessed and documented with close-up photography. Photographs of the face are taken with a Nikon M3 digital camera. Photographs are evaluated using an image analysis program, Optimas 6.5. Lines and wrinkles are assessed by examining changes in the Integrated Optical Density (IOD) before and after product application. A decrease in IOD represents a decrease in fine lines and wrinkles.

Standard Procedure for Using the Optimas 6.5 Program:

Instrumentation:
1. Fuji S2 or Nikon M3 digital camera.
2. Computer station with Optimas 6.5 (Media Cybernetics, San Diego, Calif.) Image Analysis Program.

Photography:
1. Panelists heads are placed in a head rest to insure reproducibility of positioning.
2. The camera is positioned one foot from the panelist at an F stop of 32. An unpolarized photograph of the area demonstrating fine lines and/or wrinkles is taken.

Program Operation:
1. The photographs are opened and displayed in the Optimas 6.5 program.
2. Using editing tools in the Optimas 6.5 software, a region of interest (ROI) is masked on the area where lines are visible. When evaluating the lines before and after product use, the same ROI is used at each time point.
3. The program automatically segments the darker, indented areas (i.e. lines).
4. Each photograph is analyzed by the computer program for the area occupied by the lines in the segmented ROI, and a grey scale value is assigned to the area. A lower area represents the presence of fewer lines, while a higher area represents the presence of more lines. A lower grey value represents the presence of deeper lines, while a higher grey value represents the presence of less deep lines. The grey scale is shown below.

Grey Scale:

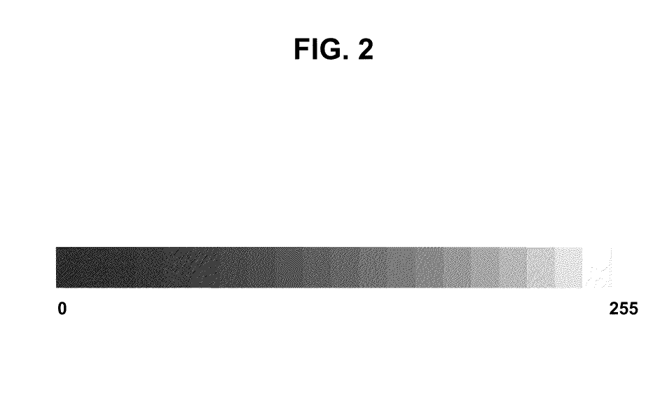

The grey scale is widely used by photographers and the scientific community as a method to assign a number value to the “lightness” or “darkness” of an image. The scale starts at zero (black), and ends at 255 (white). Everything between 0 and 255 is a shade of grey. A picture of the scale is shown in FIG. 2.

When wrinkles are photographed, a grey value is assigned to the “darkness” of each wrinkle A deeper wrinkle appears darker than a shallow wrinkle; thus, a deeper wrinkle is assigned a lower grey value.
5. Lines and wrinkles are assessed by examining changes in the Integrated Optical Density (IOD) before and after product use. IOD is equal to [(255−grey value)×area]. A decrease in IOD represents a decrease in the number and/or the depth of fine lines and wrinkles, while an increase in IOD represents an increase in the number and/or the depth of fine lines and wrinkles
6. The image analysis program is interfaced with Microsoft Excel and automatically places the values in an Excel spreadsheet.

Optical Reduction in the Appearance of Pore Size

The appearance of each panelist's pores is assessed prior to, and immediately after product application using close-up photography. The panelist's head is place in a head rest to insure reproducibility of positioning. The camera is positioned at a distance of 2 feet from the panelist with an F stop of 32. Photographs of the right and the left sides of the panelist's face are taken using a Nikon M3 digital camera. Grading is in accordance with a 10 point analog scale, and a percent change from baseline is calculated. Clinical evaluations of pore visibility are conducted by an investigator who has an extensive perceptual vocabulary, and who is experienced in scale usage and the use of standardized evaluation techniques. A standard lexicon and references for that specific parameter (i.e. a photo scale depicting the typical appearance of a “0”, of a “2”, etc., up to “10”) are used for evaluation.

$$\underline{10 point scale} \text{:}$$

$$\overline{\begin{matrix}0 & & 10 \\ no visible pores & & highly visible pores\end{matrix}}$$

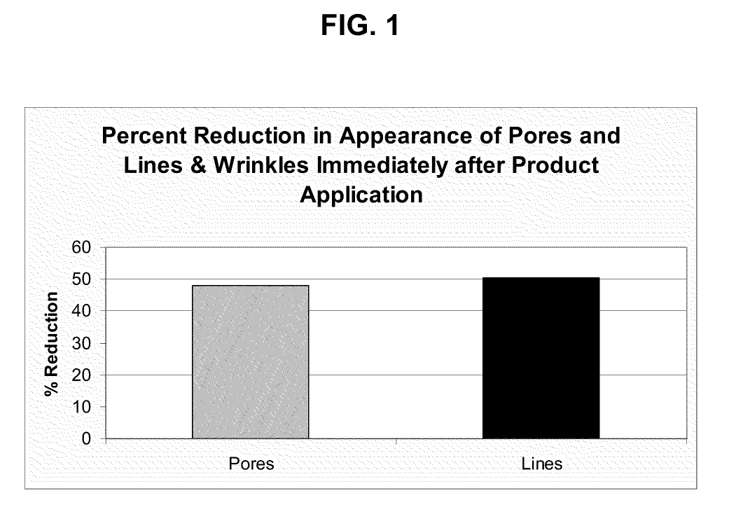

As shown in FIG. 1, the panelists demonstrated, on average, a 48% reduction in the appearance of pores and a 51% reduction in the appearance of lines and wrinkles immediately after the application of the product.

### Example 3

### Composition of the Invention—Serum

TABLE 2

| | WEIGHT |
|---|---|
| MATERIALS | PERCENT |
| WATER | q.s. |
| HDI/TRIMETHYLOL HEXYLLACTONE | 10.000000 |
| CROSSPOLYMER//SILICA | |
| DIMETHICONE | 5.882250 |
| SILICA | 5.000000 |
| ISODODECANE | 3.800000 |
| POLYSILICONE-11 | 3.632750 |
| GLYCERIN | 2.261250 |
| BUTYLENE GLYCOL | 2.200500 |
| BUTYROSPERMUM PARKII (SHEA BUTTER) | 1.550000 |
| EXTRACT | |
| HYDROGENATED LECITHIN | 1.505000 |
| PEG-11 METHYL ETHER DIMETHICONE | 1.000000 |
| PENTYLENE GLYCOL | 1.000000 |
| POLYGLYCERYL-3 DISILOXANE DIMETHICONE | 0.999800 |
| AMMONIUM ACRYLOYLDIMETHYLTAURATE/VP | 0.700000 |
| COPOLYMER | |
| MICA/TITANIUM DIOXIDE | 1.000000 |
| PROPYLENE GLYCOL DICAPRATE | 0.549500 |
| LECITHIN | 0.505000 |
| SUCROSE | 0.500000 |
| TOCOPHERYL ACETATE | 0.500000 |
| ETHYLHEXYLGLYCERIN | 0.497500 |
| HELIANTUS ANNUUS (SUNFLOWER) SEEDCAKE | 0.405000 |
| PHENOXYETHANOL | 0.393843 |
| CAPRYLYL GLYCOL | 0.363275 |
| CYCLOPENTASILOXANE | 0.250000 |
| PHOSPHOLIPIDS | 0.250000 |
| CAFFEINE | 0.200000 |
| SIGESBECKIA ORIENTALIS (ST. PAUL'S | 0.200000 |
| WORT) EXTRACT | |
| POLYSORBATE 40 | 0.200000 |
| DIMETHICONE CROSSPOLYMER-3 | 0.200000 |
| GLYCERYL POLYMETHACRYLATE | 0.159980 |
| DISODIUM EDTA | 0.101500 |
| HYDROLYZED FISH (PISCES) COLLAGEN | 0.100000 |
| SODIUM DEHYDROACETATE | 0.100000 |
| GLYCINE MAX (SOYBEAN) POLYPEPTIDE | 0.098832 |
| ALGAE EXTRACT | 0.098000 |
| LAURETH-23 | 0.084500 |
| PROPYLENE GLYCOL DICAPRYLATE | 0.084000 |
| HEXYLENE GLYCOL | 0.066050 |
| LAURETH-12 | 0.060000 |
| CHOLESTEROL/POTASSIUM SULFATE | 0.050000 |
| LAURETH-4 | 0.042500 |
| ADENOSINE PHOSPHATE | 0.040000 |
| AMINOPROPYL ASCORBYL PHOSPHATE | 0.040000 |
| PEG-8 | 0.040000 |
| ANTHEMIS NOBILIS (CHAMOMILE) | 0.039436 |
| PERSEA GRATISSIMA (AVOCADO) OIL | 0.036000 |
| CUCUMIS MELO (MELON) FRUIT EXTRACT | 0.030000 |
| HORDEUM VULGARE (BARLEY) EXTRACT\ | 0.030000 |
| EXTRAIT D'ORGE | |
| ALGIN | 0.025000 |
| SODIUM CITRATE | 0.025000 |
| SODIUM HYALURONATE | 0.020000 |
| TROMETHAMINE | 0.020000 |
| CHLORPHENESIN | 0.015000 |
| POTASSIUM SORBATE | 0.012234 |
| DISODIUM DISTYRYLBIPHENYL DISULFONATE | 0.011600 |
| CUCUMIS SATIVUS (CUCUMBER) FRUIT | 0.007500 |
| EXTRACT | |
| ACETYL HEXAPEPTIDE-8 | 0.005000 |
| SERINE | 0.005000 |
| FISH (PISCES) ATELOCOLLAGEN | 0.005000 |
| SORBIC ACID | 0.003750 |
| TOCOPHEROL | 0.002700 |
| ARTEMIA EXTRACT | 0.001500 |
| POLYGONUM CUSPIDATUM ROOT EXTRACT | 0.001000 |
| CHAMOMILLA RECUTITA (MATRICARIA) | 0.000967 |
| SODIUM CHLORIDE | 0.000900 |
| CAMELLIA SINENSIS (WHITE TEA) LEAF | 0.000700 |
| EXTRACT | |
| ASCORBYL TOCOPHERYL MALEATE | 0.000500 |
| ROSMARINUS OFFICINALIS (ROSEMARY) | 0.000500 |
| LEAF EXTRACT | |
| NORDIHYDROGUAIARETIC ACID | 0.000500 |
| TOTAL | 100% |

Numerous variations and modifications of the invention will become readily apparent to those familiar with cosmetic products. Accordingly, the scope of the invention should not be construed as limited to the specific examples described, as those examples are presented herein only as being illustrative of the many formulations possible according to the invention.

What is claimed is:

1. A silicone-in-water emulsion for reducing the appearance of one or more of fine lines, wrinkles and the size of pores in the skin, comprising (a) at least one silicone, comprising at least one silicone elastomer; (b) at least one silica (c) a film-forming component; and (c) water.

2. The emulsion according to claim 1, consisting essentially of (a), (b), (c) and (d).

3. The emulsion according to claim 1, wherein the at least one silicone is selected from the group consisting of volatile silicones, non-volatile silicones, silicone elastomers, silicone gums, silicone waxes, and linear silicones.

4. The emulsion according to claim 1, in which the at least one silicone elastomer is selected from the group consisting of dimethicone//dimethicone/vinyl dimethicone crosspolymer, phenyl trimethicone//dimethicone/phenyl vinyl dimethicone crosspolymer, cyclopentasiloxane//dimethicone/vinyl dimethicone crosspolymer, dimethicone/polysilicone-11, cyclopentasiloxane/polysilicone-11, phenyl trimethicone/polysilicone-11, cyclomethicone/polysilicone-11/petrolatum, cyclomethicone/polysilicone-11, stearoxy methicone/dimethicone copolymer, a mixture of water/propylene glycol/cyclomethicone D5/polysilicone-11/butylenes glycol/glycerine/isohexadecane/ammonium polyacryloyldimethyl, a mixture of butylenes glycol/water/dimethicone/polysilicone-11/glycerin/isohexadecane/ammonium polyacryloyldimethyl taurate/retinyl palmitate, dimethicone/polysilicone-11/cyclomethicone cyclomethicone/trimethylsiloxysilicate, and vinyldimethicone/methicone silsesquioxane crosspolymer.

5. The composition according to claim 4, in which the silicone elastomer comprises vinyldimethicone/methicone silsesquioxane crosspolymer.

6. The emulsion according to claim 1, in which the at least one silica is selected from the group consisting of silica, silica silylate, silica/triethoxycaprylsilane, silica/methicone, silica/dimethicone, silica/isononyl isonanoate, silica/octyldodecyl stearoyl stearate/isononyl isononanoate, silica//methoxy amodimethicone silsesquioxane copolymer, silica/ultramarines/kaolin/PVP/water, and hexyldecyldiisocyanate/trimethylol hexylactone crosspolymer//silica.

7. The emulsion according to claim 7, wherein the silica comprises a spherical silica, hexyldecyldiisocyanate/trimethylol hexylactone crosspolymer//silica or a mixture thereof.

8. The emulsion according to claim 7, wherein the silica comprises a spherical silica and hexyldecyldiisocyanate/trimethylol hexylactone crosspolymer//silica.

9. The silicon-in-water emulsion according to claim 1, wherein the film-forming component comprises algin, serine, and atelocollagen.

10. The silicon-in-water emulsion according to claim 1, wherein the film-forming component comprises a water-soluble polymer of 2-methacryloyloxy ethyl phosphorylcholine.

11. The emulsion according to claim 1, which comprises in the range of from about 5 to about 40 weight percent silicone, in the range of from about 1 to about 40 weight percent silica, and in the range of from about 0.01 to about 20 weight percent of the film-forming component, by total weight of the emulsion.

12. The emulsion according to claim 11, which comprises in the range of from about 10 to about 30 weight percent of silicone, in the range of from about 10 to about 30 weight percent silica, and in the range of from about 0.1 to about 10 weight percent of the film-forming component, by total weight of the emulsion.

13. The emulsion according to claim 12, which comprises in the range of from about 10 to about 20 weight percent silicone, in the range of from about 10 to about 20 weight percent silica, and in the range of from about 1 to about 5 weight percent of the film-forming component, by total weight of the emulsion.

14. The emulsion according to claim 1, which comprises in the range of from about 5 to about 40 percent of the silicone elastomer, by total weight of the emulsion.

15. The emulsion according to claim 1, which comprises in the range of from about 10 to about 20 percent of the silicone elastomer, by total weight of the emulsion.

16. The emulsion according to claim 1, wherein the silicone elastomer comprises vinyldimethicone/methicone silsesquioxane crosspolymer, the silica comprises hexyldecyldiisocyanate/trimethylol hexylactone crosspolymer//silica and the film-forming component comprises algin, serine, and atelocollagen.

17. The emulsion according to claim 1, wherein the silicone elastomer comprises vinyldimethicone/methicone silsesquioxane crosspolymer, the silica comprises hexyldecyldiisocyanate/trimethylol hexylactone crosspolymer//silica and a spherical silica, and the film-forming component comprises algin, serine, and atelocollagen.

18. The emulsion according to claim 1, wherein the silica comprises hexyldecyldiisocyanate/trimethylol hexylactone crosspolymer//silica and a spherical silica, and the film-forming component comprises algin, serine, and atelocollagen.

19. The emulsion according to claim 1, further comprising a surfactant which is selected from the group consisting of silicone surfactants, non-ionic surfactants and mixtures thereof.

20. The emulsion according to claim 1, further comprising at least one cosmetically acceptable ingredient, selected from the group consisting of preservatives, skin conditioning agents, thickening agents, structuring agents, botanical extracts, humectants, vitamins, and antioxidants.

21. The emulsion according to claim 1, which is a lotion, a cream, a gel, a cream gel or a serum.

22. A method for reducing the appearance of one or more of fine lines, wrinkles and the size of pores in the skin, comprising applying to the skin in need of a reduction in the appearance of lines, wrinkles and/or pores, a cosmetically or dermatologically effective amount of a silicone-in-water emulsion comprising (a) at least one silicone comprising at least one silicone elastomer; (b) at least one silica; (c) a film-forming component; and (d) water.

23. The method of claim 22, wherein the emulsion consists essentially of (a), (b), (c), and (d).

24. The method of claim 22, wherein the at least one silicone in the emulsion selected from the group consisting of volatile silicones, non-volatile silicones, silicone elastomers, silicone gums, silicone waxes, and linear silicones.

25. The method according to claim 22, in which the at least one silicone elastomer is selected from the group consisting of dimethicone//dimethicone/vinyl dimethicone crosspolymer, phenyl trimethicone//dimethicone/phenyl vinyl dimethicone crosspolymer, cyclopentasiloxane//dimethicone/vinyl dimethicone crosspolymer, dimethicone/polysilicone-11, organopolysiloxanes, cyclopentasiloxane/polysilicone-11, phenyl trimethicone/polysilicone-11, cyclomethicone/polysilicone-11/petrolatum, cyclomethicone/polysilicone-11, stearoxy methicone/dimethicone copolymer, a mixture of water/propylene glycol/cyclomethicone D5/polysilicone-11/butylenes glycol/glycerine/isohexadecane/ammonium polyacryloyldimethyl, a mixture of butylenes glycol/water/dimethicone/polysilicone-11/glycerin/isohexadecane/ammonium polyacryloyldimethyl taurate/retinyl palmitate, dimethicone/polysilicone-11/cyclomethicone cyclomethicone/trimethylsiloxysilicate, and vinyldimethicone/methicone silsesquioxane crosspolymer.

26. The method according to claim 25, in which the silicone comprises vinyldimethicone/methicone silsesquioxane crosspolymer.

27. The method according to claim 22, in which the at least one silica is selected from the group consisting of silica, silica silylate, silica/triethoxycaprylsilane, silica/methicone, silica/dimethicone, silica/isononyl isonanoate, silica/octyldodecyl stearoyl stearate/isononyl isononanoate, silica//methoxy amodimethicone silsesquioxane copolymer, silica/ultramarines/kaolin/PVP/water, and hexyldecyldiisocyanate/trimethylol hexylactone crosspolymer//silica.

28. The method according to claim 27, wherein the silica comprises a spherical silica, hexyldecyldiisocyanate/trimethylol hexylactone crosspolymer//silica or a mixture thereof.

29. The method according to claim 28, wherein the silica comprises a spherical silica and hexyldecyldiisocyanate/trimethylol hexylactone crosspolymer//silica.

30. The method according to claim 22, in which the emulsion comprises in the range of from about 5 to about 40 weight percent silicone, in the range of from about 1 to about 40 weight percent silica, and in the range of from about 0.01 to about 20 weight percent of the film-forming component, by total weight of the emulsion.

31. The method according to claim 30, in which the emulsion comprises in the range of from about 10 to about 30 weight percent of the silicone, in the range of from about 5 to about 30 weight percent silica, and in the range of from about 0.1 to about 10 weight percent of the film-forming component, by total weight of the emulsion.

32. The method according to claim 31, in which the emulsion comprises in the range of from about 10 to about 20 weight percent of the silicone in the range of from about 5 to about 10 weight percent of the silica, and in the range of from about 1 to about 5 weight percent of the film-forming component, by total weight of the emulsion.

33. The method of claim 22, wherein the emulsion comprises in the range of from about 10 to about 20 percent of the silicone elastomer, by total weight of the emulsion.

34. The method according to claim 22, wherein the silicone elastomer comprises vinyldimethicone/methicone silsesquioxane crosspolymer, the silica comprises hexyldecyldiisocyanate/trimethylol hexylactone crosspolymer//silica and the film-forming component comprises algin, serine, and atelocollagen.

35. The method according to claim 22, wherein the silicone elastomer comprises vinyldimethicone/methicone silsesquioxane crosspolymer, the silica comprises hexyldecyldiisocyanate/trimethylol hexylactone crosspolymer//silica and a spherical silica, and the film-forming component comprises algin, serine, and atelocollagen.

36. The method according to claim 22, wherein the silica comprises hexyldecyldiisocyanate/trimethylol hexylactone crosspolymer//silica and a spherical silica, and the film-forming component comprises algin, serine, and atelocollagen.
